(19) United States
(12) Patent Application Publication
(10) Pub. No.: US 2014/0249573 A1
(43) Pub. Date: Sep. 4, 2014

(54) METHODS AND DEVICES FOR OCCLUDING BLOOD FLOW TO AN ORGAN

(71) Applicant: A.A. CASH TECHNOLOGY LTD., Holon (IL)

(72) Inventor: Amir Arav, Tel Aviv (IL)

(73) Assignee: A.A. CASH TECHNOLOGY LTD., Holon (IL)

(21) Appl. No.: 14/346,249

(22) PCT Filed: Sep. 19, 2012
(86) PCT No.: PCT/IL12/50373

§ 371 Date: Mar. 20, 2014

Related U.S. Application Data
(60) Provisional application No. 61/536,600, filed on Sep. 20, 2011.

(51) Int. Cl. A61B 17/12

(52) U.S. Cl. CPC A61B 17/12009
USPC 606/202; 606/201

(57) ABSTRACT

A method for protecting at least one gonad from a blood borne cytotoxic drug, the method comprising reducing blood flow to at least one gonad of a patient undergoing cytotoxic treatment for an occlusion time interval, and allowing blood flow to the at least one gonad to resume after the occlusion time interval. Optionally the method is performed to protect an ovary during chemotherapy treatment.

### FIELD OF THE INVENTION

The invention, in some aspects thereof, relates to the field of surgical implantation of blood occluding devices.

### BACKGROUND OF THE INVENTION

Many drugs are carried by a patient's blood system from a site of administration to organs and tissues throughout the body. Some of these organs and tissues may be targets of treatment, while others might not, and the drugs might have undesired side effects on organs and tissues which are not intended for treatment. For example, some chemotherapy drugs are toxic to reproductive cells and organs. Known side effects of chemotherapy in young women include sterility and/or premature menopause. Similarly, men undergoing chemotherapy often suffer damage to their natural fertility, for example chemotherapy induced oligospermia.

Men undergoing chemotherapeutic treatments often preserve their fertility through sperm banking. To date, the solutions offered to women include cryopreservation of embryos or, albeit still investigational, freezing of oocytes or preservation of ovarian tissue. Both techniques require a delay in cancer treatment for at least one month, which is not an option for some patients. Other proposed solutions involve cryopreservation of ovarian tissue, for example as ovarian cortical slices or as a whole ovary.

### SUMMARY OF THE INVENTION

The invention in some aspects thereof relates to reducing exposure of an in vivo organ (for example a gonad, such as an ovary) to a blood borne drug, by reducing (or even preventing) blood flow to the organ at a time when the blood borne drug is in the blood system of a patient.

For example, a controllable blood occluding device may be implanted in a patient in association with at least one blood vessel supplying blood to and/or taking blood from an organ before the patient undergoes chemotherapy. During drug administration and/or thereafter, the device may be controlled to reduce or even prevent blood flow by occluding the blood vessel (partially or completely). Such occlusion may be terminated at a later time, for example before any significant irreversible ischemic damage is caused to the organ. Optionally, occlusion is performed intermittently during the time interval when the blood borne drug is in the blood system of a patient, thereby reducing (but not preventing) exposure to the drug.

There is therefore provided in accordance with some embodiments of the invention a method for protecting at least one gonad (e.g. ovary or testes) from a blood borne cytotoxic drug, the method comprising:
reducing blood flow to at least one gonad of a patient undergoing cytotoxic treatment for an occlusion time interval; and
allowing blood flow to the at least one gonad to resume after the occlusion time interval.

The occlusion time interval may last any period of time from minutes, to hours and even longer. Optionally, the occlusion time interval lasts 24 hours or less or even 12 hours or less.

Optionally, during at least a portion of the occlusion time interval, blood flow to and/or from the at least one gonad is stopped. Optionally, blood flow is reduced but not completely stopped, for example by intermittent stopping of blood flow and/or by intermittent or constant partial occlusion of blood flow. Optionally, partial occlusion comprises reducing the diameter of one or more blood vessels. Optionally, partial occlusion comprises stopping and/or reducing the rate of blood flow through some, but not all blood arteries and/or veins of the at least one gonad.

The occlusion time interval may be timed according to the concentration of the blood borne drug (e.g. a chemotherapeutic drug) in the blood and/or the potential damage from the drug at a given concentration. Thus the occlusion time interval may overlap, partially or completely, a time when the blood borne cytotoxic drug is present in the blood system of the patient. Optionally, the occlusion time interval covers at least the period of time where the cytotoxic drug is at a concentration in the blood that is sufficient to cause damage to a gonad.

The occlusion time interval may begin when the cytotoxic drug is being administered to the patient, or even before that. In fact, the method may comprise administering the blood borne cytotoxic drug to the patient for drug administration time interval, and optionally the occlusion time interval covers a period of time after the end of the drug administration time interval. Optionally, the occlusion time interval spans a period of covering at least a portion of the drug administration time interval and ending at a time that is at least equal to the drug's T₅₀ measured from the end of the drug administration time interval or for a point of time when the drug is at its peak concentration in the patient's blood.

Optionally, ischemia reducing treatment is applied to the at least one gonad before, during and/or after the occlusion time interval. For example, this may comprise infusing the at least one gonad with an ischemia reducing agent (e.g. an ischemia reducing agent comprising or consisting of an antioxidant and/or an anticoagulant). Optionally, applying an ischemia reducing treatment to the at least one gonad comprises cooling a portion of the blood member binding element and/or allowing intermittent and/or partial blood flow to the gonad.

Optionally, the method comprises implanting a device in association with a blood vessel that supplies blood to and/or takes blood from the at least one gonad, and reducing blood flow and allowing blood flow to resume are perfotbrmed, at least in part, using the implanted device.

In some embodiments of the invention, a system for protecting at least one gonad from a blood borne cytotoxic drug is disclosed, the system comprising:
an implantable device comprising a blood vessel binding member comprising a gap having an adjustable width and configured for accepting through it at least one blood vessel supplying blood to and/or taking blood from the at least one gonad; and
a control interface for adjusting the gap such that a pressure exerted on the at least one blood vessel is changed.

The system may be configured to perform at least a part of the disclosed methods. Optionally, the at least one gonad comprises an ovary and the blood vessel binding member is sized and shaped to be positioned between the ovary and a uterus wall.

Optionally, the blood vessel biding member comprises an inflatable portion. In such instances, the control interface may comprise a port usable for inflating and deflating the inflatable surface portion. Optionally, the system further comprises a fluid reservoir for moving a fluid to and from the inflatable portion. Optionally, the fluid reservoir is implantable.

Additionally or alternatively, the system comprises a controller (e.g. implantable and/or external) for controlling the adjusting of the gap. The controller may be configured control the adjusting using remote communication (inside the body and/or from the outside).

Optionally, the controller is configured to reduce the gap for an occlusion time interval and increase the gap at the end of the occlusion time interval. Optionally, the controller is configured to intermittently reduce and increase the pressure exerted on the at least one blood vessel during the occlusion time interval.

Optionally, the controller is configured to limit the occlusion time interval to a period selected according to data relating to administration of a cytotoxic drug. For example, a medical practitioner may input to the controller via a user interface information relating to the drug and/or the patient and/or the intended dose and other drug related or treatment specific and/or patient specific parameters and a processor associated with the controller would then provide an operation protocol (by calculation and/or by accessing a database). Optionally, the controller is configured to perform an operation protocol selected from a plurality of selectable operation protocols, and selection may be made based on the input.

Optionally, the system comprises one or more sensors for providing input to the controller. For example, the system may comprise one or more drug administration sensors configured for being associated with a drug administering device and for providing data relating to the administration of a cytotoxic drug. In such case the controller is configured to receive data from the drug administration sensor, and to control the adjustment based on the data.

Additionally or alternatively, the system may comprise one or more ischemia sensors for sensing one or more ischemia related parameters at the at least one gonad. In such case the controller is configured to receive the parameter(s) from the ischemia sensor, and to control the adjustment based on the parameter.

Additionally or alternatively, the system may comprise one or more blood flow related sensors for sensing a blood flow related measure relating to the at least one gonad, and the controller may be configured to receive the measure and to control the adjustment based on the measure.

A device for occluding a blood vessel, which may be used as part of the system, is provided. The device comprising a blood vessel binding member, which in turn comprises:
an open-frame shaped member defining a gap for accepting at least one blood vessel;
an adjustable frame-completing member configured to form, together with the open-frame shaped member, an essentially closed frame around the at least one blood vessel; and
an adjustable gap filling member positioned to adjust the gap within the frame such that a pressure exerted on the at least one blood vessel is changed.

The frame-completing member may include a moveable bar shaped element and/or an extendable portion. Once formed, the essentially closed frame may be capable of providing radial support to an adjustable gap filling member positioned within the frame.

Optionally, the open-frame shaped member includes a curved portion defining a portion of the frame shape. Optionally the adjustable gap filling member is attached to the open-frame shaped member, and optionally comprises an inflatable portion. At times, the open-frame shaped member includes a furrow shaped portion positioned around at least a portion of the inflatable portion.

The inflatable portion may be connected to a supplying port by an elongated tube, and optionally to a fluid reservoir, in which case, the flow of fluid between the inflatable portion and the reservoir may be controlled by a controller.

Optionally, the blood vessel binding member is implantable and/or extractable by a laparoscopy, for example through a single trocar. The device may comprise a shaft for directing the device in the body during implantation, the shaft being removable from the device. The shaft may be attached to the device by a lock/release structure, such that the shaft is separable only when the frame is closed. The shaft may further be attached to the device such that it may guide the implantable portion to its implantation location while the implantable portion is kept in an open frame configuration. The shaft may comprise a plunger for causing an implantable portion to shift between a closed frame configuration and an open frame configuration. Optionally, the shaft comprises a lock/release structure for attachment to an implantable portion in a closed frame configuration, for use for example, in extracting the device.

In some embodiments of the invention, a device for occluding a blood vessel is provided, which may be used as part of the system. This device comprises a blood vessel binding member which comprises:
a frame shaped member defining a gap for accepting at least one blood vessel and having at least an expanded configuration and a collapsed configuration; and
an adjustable gap filling member associated with the frame shaped member and positioned to adjust the gap within the frame such that a pressure exerted on the at least one blood vessel is changed.

Optionally, the frame shaped member is configured such that a whole ovary may be passed through the gap when the frame shaped member is in an expanded configuration without causing significant damage to the ovary. Optionally, the whole ovary may be passed through the gap while the ovary is within the body and connected to the body by at least a portion of a ligament and at least one blood vessel.

The frame shaped member may comprise a plurality of linked rigid units with interspersed separators. At least one separator may include a hinge structure, and or a flexible unit. The essentially closed frame may be capable of providing radial support to the adjustable gap filling member positioned within the frame, for example when in an expanded configuration.

Optionally the device may comprise a frame shaped inflatable portion associated with the open-frame shaped member such that inflation of the frame shaped inflatable portion causes the open-frame shaped member to assume the expanded configuration. This frame shaped inflatable portion may also be the adjustable gap filling member.

Optionally, the frame shaped member includes a furrow shaped portion positioned around at least a portion of the inflatable portion. The inflatable portion may be connected to a supplying port by an elongated tube and/or to a fluid reservoir. The flow of fluid between the inflatable portion and the reservoir may be controlled by a controller.

In some embodiments, the adjustable gap filling member may be in at least one of three inflation statuses comprising:
a collapse inflation status;
a partially inflation status, wherein the gap filling member holds the open-frame shaped member in the expanded configuration; and
a high inflation status wherein the gap filling partially fills the gap such that an ovary may not pass through the gap and pressure is exerted on the at least one blood vessel.

Optionally, the blood vessel binding member is implantable and/or extractable by a laparoscopy. Optionally, this may be performed through a single trocar. During implantation and/or extraction, the device may comprise a shaft for directing the device in the body, the shaft being removable from the device. The shaft may be attached to the device through a lock/release structure which prevents unintentional release of the shaft from the device.

While the above description was provided mainly in the context of one or more gonads, it is envisioned that this method may be applied, in all or some of the disclosed options, to other organs where it is desired to temporarily prevent blood flow into the organ, thereby to protect the organ from a blood borne drug. Such a method may comprise:
reducing blood flow to at least one organ of a patient undergoing drug treatment for an occlusion time interval; and
allowing blood flow to the at least one organ to resume after the occlusion time interval.

Optionally, this method includes administering the blood borne drug to the patient for a drug administration time interval.

This Summary of the Invention is provided solely to highlight some aspects of the invention. Further details and/or alternatives are provided in the Detailed Description of Exemplary Embodiments, and the Summary of the Invention is not to be used to limit the scope of the claimed subject matter.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 schematically depicts a method of protecting a gonad according to certain embodiments of the invention;

FIG. 2 schematically illustrates devices according to certain embodiments of the invention, placed in association with at least one blood vessel supplying blood to and/or taking blood from an ovary;

FIGS. 3A-3C schematically depict devices according to some embodiments of the invention, having a frame shaped member and a shaft;

FIGS. 4A-4C schematically depict a device according to some embodiments of the invention. FIG. 4A shows a perspective view of the device; FIG. 4B shows a perspective view of some implantable components of an implantable portion of the device; and FIG. 4C shows a perspective view of some components of the shaft;

FIGS. 5A and 5B provide enlarged rotated views of a distal portion of a device outlined by dashed line A in FIG. 4A, where FIG. 5A depicts a perspective view and FIG. 5B depicts a partially exploded view of FIG. 5A;

FIG. 5C provides a view of portions of the lock/release mechanism of the device of FIGS. 5A and 5B;

FIGS. 6A-6F depict a cross section through the distal portion of a device as shown in FIG. 6A at different stages during implantation and extraction of a device according to some embodiments of the invention. FIG. 6A shows the device with a shaft attached and the frame shaped member open; FIGS. 6B & 6C depict the steps of moving the shape-completing member to a position where the frame shape is closed and enabling separation of the shaft from the frame shaped member; FIGS. 6D & 6E depict steps of detaching shaft members from the closed frame shaped member; and FIG. 6F depicts the frame shaped member with the frame closed after the shaft was detached; and

FIGS. 7A-7E depict a perspective view of a device according to some embodiments hereof. FIG. 7A depicts the device in a collapsed configuration for example as it may be while crossing through a trocar; FIG. 7B depicts the device in expanded configuration showing a fully opened gap; and FIG. 7C shows the device in an expanded configuration showing a partially closed gap. FIG. 7D provides an enlarged view of the collapse blood vessel binding member as seen in FIG. 7A and FIG. 7E provides an enlarged view of the expanded blood vessel binding member as seen in FIG. 7B.

### DETAILED DESCRIPTION OF EMBODIMENTS

In the following description components that are common to more than one figure may be referenced by the same reference numerals.

In addition, unless specifically noted, embodiments described or referenced in the present description can be additional or alternative to any other embodiment described or referenced therein.

The invention, in some aspects thereof, relates to methods and devices for protecting an organ from a blood borne drug, for example, one or more cytotoxic or gonadotoxic drugs being present in the body in connection for cytotoxic treatment, such as in chemotherapy. The drug(s) may be administered to the patient and/or produced by the patient in connection with a treatment. As used herein, cytotoxic or gonadotoxic drugs may include one or more of drugs used to treat cancer, including for example alkylating drugs, including cyclophosphamide, capecitabine, fluorouracil, doxorubicin, paclitaxel, and docetaxel.

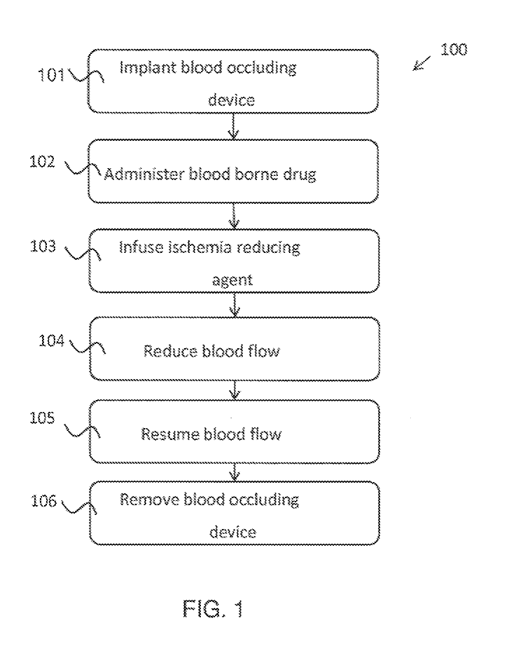

Therefore, in some embodiments of the invention, a method as schematically depicted in FIG. 1 is disclosed for protecting at least one organ of a patient undergoing cytotoxic treatment (e.g. a gonad) from a blood borne cytotoxic drug, the method comprising reducing blood flow to the organ for an occlusion time interval, and allowing blood flow to the at least one gonad to resume after the occlusion time interval. This is shown for example in FIG. 1, as steps 104 and 105 of method 100. The time interval between reducing blood flow and allowing it to resume is the occlusion time interval. As seen in FIG. 1, one or more additional optional steps may be performed in connection with some embodiments hereof.

In some cases, the method is performed by using a blood occluding device having a blood vessel binding member according to some embodiments of the invention. In some embodiments, the device may be a controllable implanted device according to some aspects of the invention, as described in detail below. In some embodiments, the device may be operated manually to control blood flow. In some embodiments, the device is controlled automatically, at least partially.

Accordingly, method 100 may include an implantation step 101, wherein a blood occluding device is implanted in a patient. The device may then be operated in one or more occasions, as needed. In some embodiments the implanted device may be removed (as depicted in optional step 106). In some embodiments step 101 and/or step 106 may be performed by laparoscopy. In some embodiments this laparoscopy may be performed via a single trocar. Optionally implantation step 101 comprises exposing or partially exposing a portion of the at least one blood vessel, and/or at least partial separation of the at least one blood vessel from connective tissue (e.g. a ligament or portion thereof) so as to allow positioning of a blood vessel binding member.

In some embodiments, the blood vessel binding member of the device comprises a gap for accepting a blood vessel and an adjustable gap filling member for adjusting the size of the gap and/or pressure exerted by the device on the blood vessel, thereby controlling blood flow to and/or from the organ. The gap filling member may consist of, or comprise, an inflatable portion (sometimes also called an inflatable member).

Optionally, during implantation, patient specific inflation statuses may be defined for an inflatable portion of the device is, for example by defining two or more of:
(a) a degree of pressure (or range) that is needed to hold the device in place without significantly affecting blood tlow through the at least one blood vessel;
(b) a degree of pressure (or range or minimal value) that is needed to completely block blood flow to the organ; and
(c) one or more interim degrees (or ranges) of pressure that would affect blood flow (or reduce the size of the gap) to a known degree.

Such values may then be used by a medical practitioner to set the device at a desired inflation status (e.g. by having a marked setting or inflating/deflating the device by a known amount of fluid.

In some embodiments, the method of the invention further includes a step 102 of administering a blood borne (potentially cytotoxic) drug. This may be performed orally or intravenously or by injection or by any other method known in the art. In some embodiments, the cytotoxic drug is produced by the patient as part of a treatment where a drug is activated, e.g., by the body, for example via metabolism at the liver and/or irradiation, and/or as a result of a combination between two or more drugs. The period during which a drug is being administered (e.g. when it is drip fed intravenously), is termed the drug administration time interval.

As known in the art, a cytotoxic blood borne drug typically reaches a peak concentration in the blood, and then reduces in concentration due to biological processes (e.g. secretion and/or metabolism) and/or natural decay. The drug's toxicity is often proportional to the drug's concentration in the blood. Accordingly, to have maximal protection from undesired drug effect, the occlusion time may be set to begin before or at the time when the drug is present in the blood system and end at a time when the drug is no longer cytotoxic to the organ or portion thereof. For example, in some embodiments, administering the drug (step 102), is performed only after reduction of blood flow is achieved (e.g. step 104). Alternatively or additionally, the occlusion time interval may cover a period of time being after the end of the drug administration time interval.

However, as blood flow in itself is important for the organ's survival and function, it may be desired to reduce the occlusion time interval to only partially overlap the time that the drug is present in the blood system of the patient. For example, the occlusion period of time may be selected to cover at least (or only) a period of time where the cytotoxic drug is at a concentration in the blood that is sufficient to cause damage to the organ (e.g. gonad). In some embodiments, the occlusion time interval may be set to span a time interval covering at least a portion of the drug administration time interval and ending at a time that is at least equal to the drug's T₅₀ measured from the end of the drug administration time interval or from the time when the drug begins to reduce from a peak concentration in the patient's blood. As used herein, a drug's T₅₀ is the time interval required for a drug's concentration to reduce in a patient's blood system to half its concentration or amount as measured at the beginning of the time interval. In some embodiments, the occlusion time interval may be set for example to last 24 hours or less, 12 hours or less, 6 hours or less or even 2 hours or less in total or from the end of a peak in the concentration of the drug in a patient's blood. At times, the occlusion time interval is in the range of 30-60 minutes (e.g. if the cytotoxic drug is administered briefly and has a short half-life in the blood). In some embodiments, the occlusion time interval may be set for example to last at least 2 hours, at least 6 hours, at least 12 hours or even at least 24 hours in total or from the end of a peak in the concentration of the drug in a patient's blood.

During the occlusion time interval, blood flow to the organ is reduced. This may be performed for example by stopping blood flow completely for at least a portion of the occlusion time interval. Additionally or alternatively blood flow may be reduced but not stopped for at least a portion of the occlusion time interval. For example, blood flow may be stopped and resumed intermittently (e.g. allowing 1-60 minute of blood flow in every 5-120 minutes). Additionally or alternatively blood flow may be reduced (e.g. by reducing the diameter of a blood vessel supplying blood to the organ or taking blood therefrom). Accordingly, in at least a portion of the occlusion time interval, blood flow may be reduced on average by at least 30%, at least 50% or even at least 80%. In fact, during the occlusion time interval the method may include a combination of one or more time intervals wherein either blood is allowed to flow freely or blood is allowed to flow at a reduced rate or blood flow is completely blocked.

In some embodiments, the reduction in blood flow is controlled in correlation to the concentration in the blood of the cytotoxic drug, and/or based on its potential damage and/or based on the organs sensitivity to ischemia in view of and during the occlusion time interval. For example, blood flow may be completely stopped or stopped to a high degree (e.g. allowing 10% blood flow or less or 25% or less or just 50% or less blood flow) for a first period of time beginning as soon as the drug reaches a minimal hazardous concentration or at a time before that, and until such time as the drug is expected to have reduced by at least a given amount (e.g. 10% or 25% or even 50%) from its peak concentration. At that time, blood flow may be maintained at a reduced rate (>0) which may be maintained until the end of the occlusion time interval or gradually increase until such time. In some embodiments, the first period of time may include interim time intervals wherein blood is allowed to flow at a greater rate in order to reduce the hazard of ischemia. In some embodiments, blood flow rate is controlled based on a balance between the hazard of ischemia and the hazard of the cytotoxic drug.

In some embodiments, ischemia reducing treatment may be applied to the organ (or the patient) before, during and/or after the occlusion time interval. In some embodiments an ischemia reducing treatment comprises use of an ischemia reducing agent. Such agent may serve to reduce, prevent or even reverse ischemic damage. In some embodiments the organ to be protected is infused with the ischemia reducing agent and/or the agent is otherwise provided (e.g. intravenously or orally or by injection, etc.).

In some embodiments, the ischemia reducing treatment is applied periodically before, during and/or after the occlusion time interval. For example, every 1 in the minutes, or between 10-20 minutes every 1-2 hours, etc.

In some embodiments, ischemia reducing treatment may comprise cooling a portion of the blood vessel binding member, for example by cooling a fluid used to inflate an inflatable member thereof. This in turn may cool the organ through contact and/or through cooling blood or another fluid that is allowed to flow into the organ through contact with the device. Optionally, cooling is applied to a fluid reservoir containing the fluid that is used to inflate the inflatable member. Optionally the fluid is cooled is to a degree between 0° C. and 25° C. or between 0° C. and 10° C. or between 0° C. and 5° C. Optionally cooling is performed by injecting a cooled fluid into the inflatable member.

Some non-limiting examples for ischemia reducing agents include use of cooled water or based solution. In some embodiments, the ischemia reducing agent may include an antioxidant. Examples for such antioxidants include one or more vitamins (e.g. vitamin C) and/or one or more polyphenols (e.g. Epigallocatechin gallate (EGCG) and/or Edaravone).

In some embodiments, the patient's own blood is used as an ischemia reducing agent, by allowing partial blood flow and/or intermittent blood flow to occur during at least a portion of the occlusion time interval. In some embodiments, blood is cooled before entering the organ.

In some embodiments, blood flow rate is controlled through feedback from the organ and its ischemic condition. For example the organ may be observed during occlusion (continuously or intermittently) and as soon as coloration changes above a predetermined threshold, blood flow is increased and/or a notification is made. This may be performed for example by laparoscopy image tool, and may be operated by a medical practitioner and/or by a controller applying image analysis to acquired images of the organ by spectroscopy.

One prominent example for a method according to some embodiments of the invention is the protection of reproductive organs and/or tissues and/or cells from a blood borne gonadotoxic drug. Such methods may be applied to male patients (i.e. protection testes) and female patients (i.e. protecting ovaries). In these cases, blood occlusion may be applied to one or both gonads. In some cases this may be combined with other methods to preserve hormonal balance and/or fertility, as are known in the art.

For example, the gonad(s) may be protected essentially as described above or below only after sperm/ova/embryos were harvested and preserved. Another example includes extracting one gonad (e.g. ovary) for cryopreservation (e.g. intact or as cortical slices) and applying blood occlusion as described herein to the other gonad.

### Example 1

The following experiment was designed to demonstrate ovary resistance to prolonged blood occlusion.

The ovaries of three 5 month old female sheep were exposed by laparoscopy and blood flow to and from the ovaries was occluded for an occlusion time interval of 24.5 or 27.5 hours. In one ovary of each sheep blood flow was occluded (full block) at the ovarian artery and ovarian vein, while in the other ovary, all of the ovarian artery and vein and the uterine artery and vein were occluded.

After the occlusion time interval the ovaries were removed and fixed in Buyen solution for histology evaluation. The results are depicted in Table 1 below. In addition, the ovaries were observed for coloration changes. The results are summarized in Table 1.

Firstly, it was noted that ovaries where all four arteries and veins were occluded, maintained a natural appearance in size and coloration, wherein ovaries with blood occlusion only at the ovarian artery and vein appeared to be swollen and hemorrhagic. Secondly, it was observed that most ovaries where both arteries were occluded had more follicles, suggesting a better preservation of function. In fact, in sheep 1272, it was observed that where both arteries were occluded intact secondary and primary follicles were maintained, whereas in the other ovary, only primordial follicles remained intact.

TABLE 1

| | | No. of | Occlusion time | | No. of |
|---|---|---|---|---|---|
| Sheep | ovary | arteries | interval | Coloration | follicles/ovary |
| 1272 | left | 2 | 27.5 h | white | 39 follicles |
| | right | 1 | 27.5 h | black | 22 follicles |
| 1273 | left | 1 | 24.5 h | black | 25 follicles |
| | right | 2 | 24.5 h | white | 23 follicles |
| 1274 | left | 1 | 24.5 h | black | 6 follicles |
| | right | 2 | 24.5 h | white | 20 follicles |

### Example 2

The following experiment shows an example for reducing gonadotoxic damage according to some embodiments of the invention.

The protocol and design of all parts of this study were approved by the Animal Research Ethics Committee of Israel. Three female pigs (The Institute of Animal Research, Kibbutz Lahav, Israel) weighing 60 Kg each, were anesthetized with 10 mg/kg intramuscular ketamine hydrochloride and 4 mg/kg xylazine hydrochloride (Vetmarket, Israel). One ovary of each pig was exposed by a longitudinal midline incision. A conventional gastric band (Lap-band; Allergen, Irvine, Calif., USA) was modified according to some embodiments of the invention, to occlude blood flow to the ovaries. The band was wound around the ovarian hilum, and a zip tie was placed on the center of the band ring to reduce the diameter of the band's aperture. 10 ml saline was injected via the band's port thus completely blocking blood flow through the ovarian and uterine arteries and veins. The color and the morphology of the ovary were observed after occlusion of blood flow as well as after the bands were released. The other ovary in each pig was left unprotected (i.e. was left with natural blood flow, and thus completely exposed to chemotherapy). Heparin was administered intravenously before occlusion to prevent or reduce blood coagulation within the ovary throughout the occlusion time interval and a few hours thereafter. A control pig was left untreated.

Following occlusion of the ovarian blood supply, a gonadotoxic drug (Cyclophosphamide; Endoxan, Baxter, 1L746c, USA) was administered intravenously (IV) at a concentration of 60 mg/kg for a drug administration time interval of 1 hour. The ovaries blood supply was occluded for 16 hours after the drug administration interval (i.e. a total occlusion time interval exceeding 17 hours). The bands were then opened by drawing out the 10 ml of saline via the port.

The following day, the lymphocyte counts of the three pigs decreased to 12% of the initial level (1.2×10⁹ cells/ml) due to the cytotoxic effects of the drug.

Eight days after the chemo treatment the pigs were euthanized according to animal ethics procedures and the ovaries were removed and examined visually, removed, weighed and follicles were counted. The results are summarized in Table 2.

In two of the three animals the ovaries that were unprotected displayed visible signs of atrophy and were significantly smaller in size than the counterparts with occluded blood supply, s. The number of follicles in the unprotected ovaries ranged between 3 and 32 (compared to 45 in the control ovary). All the ovaries that were occluded (protected) during the administration of chemotherapy administration displayed size and number of follicles which was comparable to the control animal.

TABLE 2

| | No. of Follicles | | Weight of ovary (gr) | |
|---|---|---|---|---|
| Pig No. | Unprotected | Occluded | Unprotected | Occluded |
| 4563 | 3 | 12 | 1.2 | 3.6 |
| 4561 | 27 | 30 | 2.8 | 3.8 |
| 4562 | 32 | 45 | 4.2 | 5.2 |
| Control | 45 | N/A | 4 | N/A |

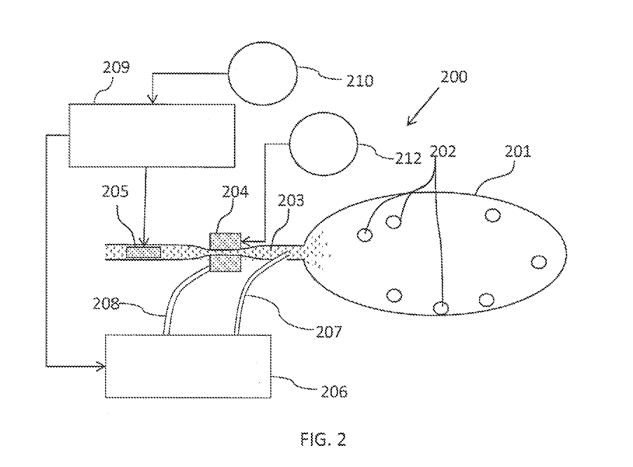

FIG. 2 schematically depicts a system 200 according to some embodiments of the invention. In this example, system 200 is shown in the context of an ovary 201. Ovary 201 comprises one or more follicles 202 that are sensitive, for example to chemotherapeutic drugs. In this example, system 200 is used to reduce blood flow to the ovary (and follicle(s)) thereby reducing their exposure to the blood borne drug.

As shown, system 200 comprises one or more blood vessel binding member 204 or intra-vessel device 205 positioned to occlude at least one blood vessel 203 that supplies blood to, or takes blood from, the organ (exemplified by an ovary 201). In this example, blood vessel binding member 204 is a device that is configured to contact the at least one blood vessel externally and to exert inward pressure to reduce the diameter of the at least one blood vessel. Examples for such implantable devices are described at further detail below. Intra-vessel device 205, on the other hand, is implantable within the at least one blood vessel (e.g. artery) and operate to allow and prevent blood flow, essentially as known in the art, with modifications discussed herein.

Optionally, the at least one blood vessel comprises a major blood vessel of the organ, namely that is responsible for more than 50% of the organ's blood supply. Optionally the at least one blood vessel comprises at least one artery and/or one vein. Optionally, the at least one blood vessel is responsible for more than 70% or even at least 85% of the organ's blood supply. Optionally, the blood vessel binding member is configured to bind, together with the at least one blood vessel, also some connective tissue associated with the blood vessel (e.g. a ligament or portion thereof).

In some embodiments, blood vessel binding member(s) 204 and/or intra-vessel device(s) 205 are controlled by a controller 209. This controller 209 may be set to regulate the operation of blood vessel binding member(s) 204 and/or intra-vessel device(s) 205, for example by reducing and/or increasing the blocking of blood flow through at least one blood vessel 203. Controller 209 may be implantable or external to the patient's body or it may system 200 may comprise both implanted and external controller components and/or controllers.

Implantable portions of system 200 may be manufactured essentially as known in the art for implantable devices and device components, including for example use of bio compatible materials, sterilization, and/or coating with or use of materials that would assist in removal of the implanted units and/or reduce negative reaction or adhesion of the patient's body to an implanted device or component. Optionally laser welding is used to produce at least some of the device components.

Blood vessel binding member(s) 204 may comprise a blood vessel binding member comprising a gap having an adjustable width and configured for accepting through it at least one artery supplying blood to ovary 201. As the gap reduces in width and closes around the at least one blood vessels changes in gap width might become small or unobserved and manifest by increasing pressure on the at least one artery.

Optionally, blood vessel binding member 204 comprises an inflatable portion which may inflate to reduce the gap and/or deflate to increase it. The inflatable portion may optionally be inflated and deflated by moving a fluid (e.g. a solution) between the inflatable portion and a fluid reservoir 211. Optionally at least one fluid reservoir 211 is implanted. Optionally at least one fluid reservoir 211 is external to the body (e.g. a syringe) and communicated with the inflatable portion through a port.

Controller 209 and/or a medical practitioner may control blood flow in operation of the system through a control interface, by adjusting the gap and such that a pressure exerted on the at least one artery is changed.

As used herein the terms adjust or adjusting mean one or more of enlargement and reduction of the size of the gap or any portion thereof and/or setting a specific size thereto. The adjusted size may depend on sensor feedback (e.g. from blood flow or blood pressure) and/or from observations by a user (e.g. retrieving ultrasound information regarding blood flow and/or observing ovary coloration changes). Adjusting may be performed to achieve a specific measure (e.g. gap size or pressure within an inflated member or rate of blood flow) and/or simply increased or decreased without specific measure, and/or shifting between a plurality of preset values. Examples for useful gap size include gaps capable of closing on and reducing the diameter of blood vessels or an amount of tissue comprising one or more blood vessels having a diameter of 5 mm or less, 3 mm or less or even 1 mm or less. In some embodiments a device may have a gap capable of being adjusted to have one or more sizes between 1 and 20 mm (or a sub range thereof).

As used herein, a control interface is an interface for causing a change in the size of the gap, including for example a user interface that allows a medical practitioner to inject fluid into at least a portion of the vessel binding member or an electronic interface for receiving an electronic control signal to actuate the adjustment of the gap (for example by moving a fluid into an inflatable member). The electronic signal may be provided by a device operated by a medical practitioner and/or from a controller (implanted or external) and may be provided by wired and/or remote communication.

In some embodiments, the blood vessel binding member 204 is configured such that it may be implanted by laparoscopy, optionally using a single trocar (e.g. a trocar being 15 mm or even 10 mm or less in diameter). In the shown example, the gap may accept one or more of the ovarian artery, ovarian vein, uterine artery and/or uterine vein. In some embodiments, blood vessel binding member 204 is sized and shaped to be positioned between the ovary and a uterus wall.

In some embodiments, system 200 comprises one or more sensors 210. One or more of sensors 210 may or may not be implanted. In some embodiments, sensors 210 may communicate directly with controller 209 or a portion thereof. Optionally, one or more sensors 210 provide information to a medical practitioner who feeds the information to controller 209 via a user interface and//or manually controls the adjustment of the gap.

One or more sensors 210 may comprise at least one drug administration sensor configured for being associated with a drug administering device and for providing data relating to the administration of a cytotoxic drug, and controller 209 may thus be configured to receive said data from and to control the adjustment based on the data. For example, sensor 210 may comprise a drip sensor associated with a device for providing intravenous chemotherapy. The drip sensor may provide data relating to beginning and/or ending of dripping and/or information relating to the rate of drug administration. Controller 209 may then be configured to control the adjustment based on the data.

One or more sensors 210 may comprise at least one ischemia sensor for sensing an ischemia related parameter at the at least one gonad, and controller 209 may then be configured to receive said parameter from the ischemia sensor, and to control the adjustment based on the parameter. For example, the ischemia sensor may be configured to sense a parameter relating to the coloration of the organ (e.g. by image acquisition and processing).

One or more sensors 210 may comprise at least one a blood flow related sensor for sensing a blood flow related measure relating to the at least one gonad, and controller 209 may then be configured to receive said measure from the blood flow related sensor, and to control the adjustment based on the measure. For example, ultrasound or Doppler readings may be performed to monitor and/or assess blood flow (e.g. periodically or throughout the occlusion time interval). Optionally, an implanted blood flow or blood pressure sensor as known in the art may be used. Optionally, a blood flow related sensor may define, and optionally set, a minimal pressure required for blocking a supply of blood to the organ. Optionally, a blood flow related sensor may define, and optionally set, a pressure or a range of pressures to be applied at one or more periods of time during the occlusion time interval.

Optionally, a controller 209 is configured to limit the occlusion time interval to a predefined period. This may occur even in devices that are configured mainly for manual operation, as a backup safety measure. The predefined period may be selected for example according to data relating the cytotoxic drug. Such data may relate to one or more of drug or treatment specific parameters that might affect the timeline and toxicity, including for example one or more of drug type/combination, concentration and time interval of drug administration, t₅₀, etc.

Optionally, controller 209 is associated with an interface for receiving at least one treatment specific parameter and the controller is configured to select an operation protocol according to the at least one treatment specific parameter. As used herein a treatment specific parameter may include one or more of data relating the cytotoxic drug and/or a patient specific parameter. For example, younger patients may be provided with a different occlusion protocol than older ones. Some patients might be known to be more sensitive to ischemia; data from previous treatments may also be used to by the controller an operation protocol.

As used herein, an operation protocol is an algorithm according to which a gap is adjusted and pressure is exerted during operation. It may include one or more of the timing and length of the occlusion time interval and the degree of reduction in blood flow at one or more points during the occlusion time interval etc. It may comprise instructions whether data from one or more sensors is to be used as a control mechanism and the relative weights attributed to types of input received from sensors. One or more of the operation protocols described herein may be applied by controller 209. Optionally controller 209 is configured to perform an operation protocol selected from a plurality of selectable operation protocols. Optionally these protocols are stored on a database in association with controller 209. Optionally specific parameters of a protocol may be set through a user interface and/or calculated by a processor associated with controller 209, based on input from one or more sensors and/or input from a user interface.

Optionally, system 200 is configured to control the application of an ischemia reducing agent. In the shown example, a flow from a reservoir 206 of an ischemia reducing agent to the organ is controlled by controller 209. Optionally, the ischemia reducing agent include the patient's own blood, in which case its flow may be regulated via blood vessel binding member(s) 204 and/or intra-vessel device(s) 205. Optionally reservoir 206 comprises a water based solution for infusing the organ through a blood vessel (e.g. via a tubing 207) by applying cooling to the blood vessel binding member (e.g. via tubing 208).

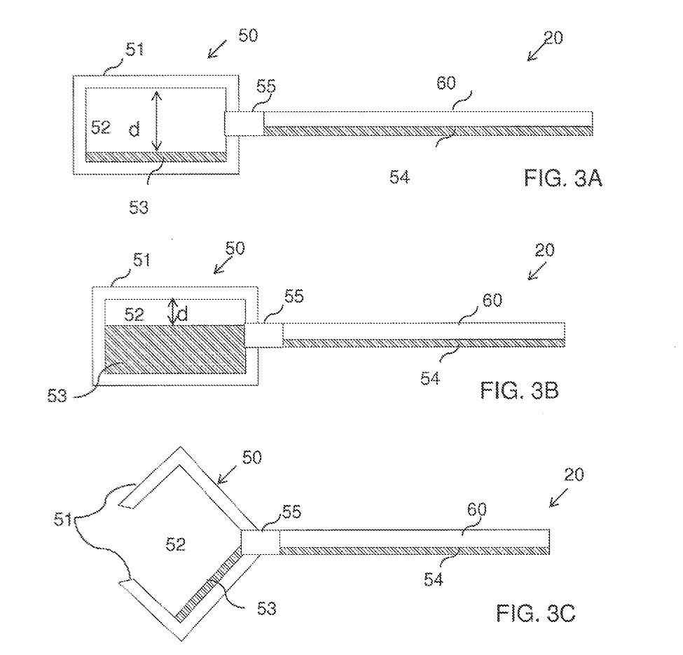

In some embodiments, system 200 may comprise a device 20 as shown schematically in FIGS. 3A-3C. Device 20 comprises a blood vessel binding member 50 and a shaft 60. As used herein, a shaft is an elongated construct having sufficient rigidity to enable the navigation the band to a desired location. Blood vessel binding member has a frame shaped portion 50 defining within it a gap 52 for accepting at least one blood vessel. Frame shaped member also comprises or is associated with an adjustable gap filling member 53. This may be of any form or shape known in the art, that is capable or reducing the size d of gap 52, such that pressure may be exerted on a blood vessel positioned within the gap may be adjusted by reducing (or attempting to reduce) the size d of gap 52. A gap filling member, as used herein, may comprise a plurality of parts or components or separate gap filling members, either linked or separate, that may be operated in connection with the adjustment of the gap and/or adjustment of the pressure exerted on the at least one blood vessel.

In the shown example, adjustable gap filling member 53 appears as an inflatable member. It is associated with tubing 54 which allows the controlled flow of a fluid to and from filling member 53. As fluid flows into adjustable gap filling member 53, it inflates and increases in size, thereby filling an increasing portion of the gap and reducing its size. When a blood vessel is positioned in the gap, the degree to inflation of adjustable gap tilling member 53 may determine the amount of pressure experienced by the vessel. This, in turn, affects the blood vessel's diameter and the rate of blood flow through it. Tubing 54 may be associated with a port through which fluid may be injected into (and/or removed from) the tubing and/or be associated with a fluid reservoir (not shown).

It is noted that adjustable gap filling member 53 is not limited to an inflatable member. It may include any movable structure that capable of being moved and/or tilted and/or extended (e.g. telescopically) in such manner as to adjust the size d of gap 52. It may include one or more of hinges, springs and/or other means for mechanical movement.

In use, frame shaped member 50 is positioned around at least one blood vessel. In some cases, this may be performed by pushing an organ through the gap such that the frame shape becomes positioned around at least one blood vessel is attached to the organ and the patient's body. In some cases, frame shaped member 50 comprises a plurality of parts at least one of which being moveable (e.g. as shown in FIG. 3C) such that frame shape 50 may be in one of at least two configurations: open (e.g. FIG. 3C) or closed (e.g. FIG. 3A). The at least one moving portion may be moved to close frame shape 50 by tilting, sliding, extending (e.g. telescopically) bending, wrapping around, or any other way. Opening and closing frame shaped member 50 may serve to engulf and/or release the at least one blood vessel.

Frame shaped member 50 optionally comprises or consists of rigid materials and/or structures that are sufficiently resilient so as to withstand the pressure required to reduce blood flow in a targeted blood vessel. Larger blood vessels may require more rigid materials. Examples for useful materials to this end include Stainless Steel or medical grade polymer such as Polycarbonate or PEEK (for the rigid frame), medical grade Silicon and for Polyethylene for example for expandable components.

In FIGS. 3A-3C, device 20 comprises a shaft 60. This shaft is depicted as an elongated rod shape. It may provide sufficient rigidity and//or resilience to guide frame shaped member 50 through the body and/or a trocar to an implantation target and/or for the removal of same. Shaft 60 may include and/or be associated with one or more lock/release structure 55. A lock release structure, is a component, or combination of component, that may provide one or more of the following functions:

1. Participate in opening and/or closing of frame shaped member 50.

2. Lock frame shaped member 50 in an open or locked position.

3. Lock and/or release frame shaped member 50 to shaft 60

Lock/release structure 55 may comprise one or more connectors for connecting to shaft 60 or a portion thereof. The connection and/or detachment of shaft 60 from connector 55 may participate in the lock/release mechanism, in which case at least a portion of shaft 60 may be deemed to be part of or comprise a lock/release structure

Cause a frame shaped member to open and/or close, for example by moving a Shaft 60 may include or be associated with a handle (not shown) at a site remote from frame shaped member 50. The handle may be used for one or more of guiding the device through a body and/or trocar, and operating one or more of the lock/release structure 55.

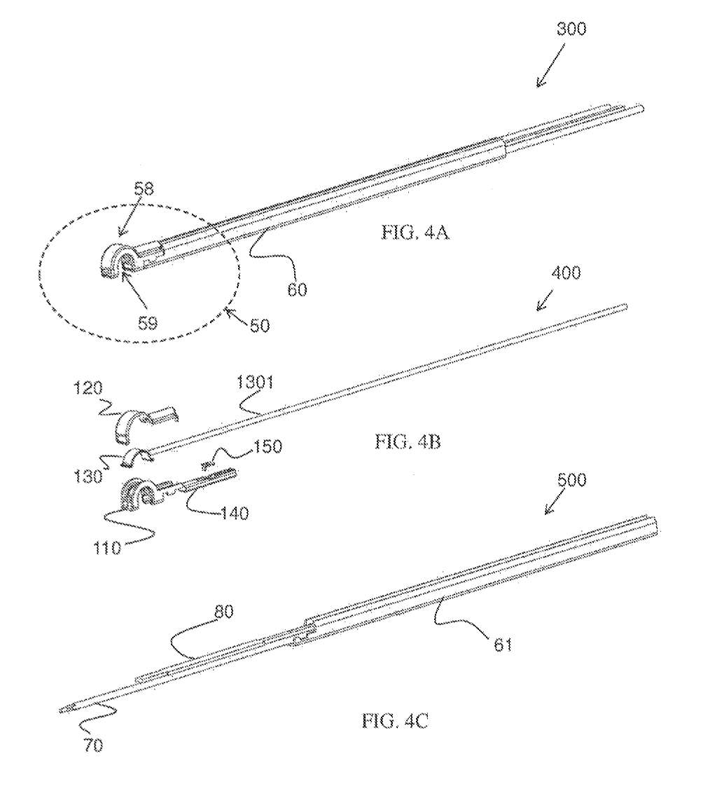

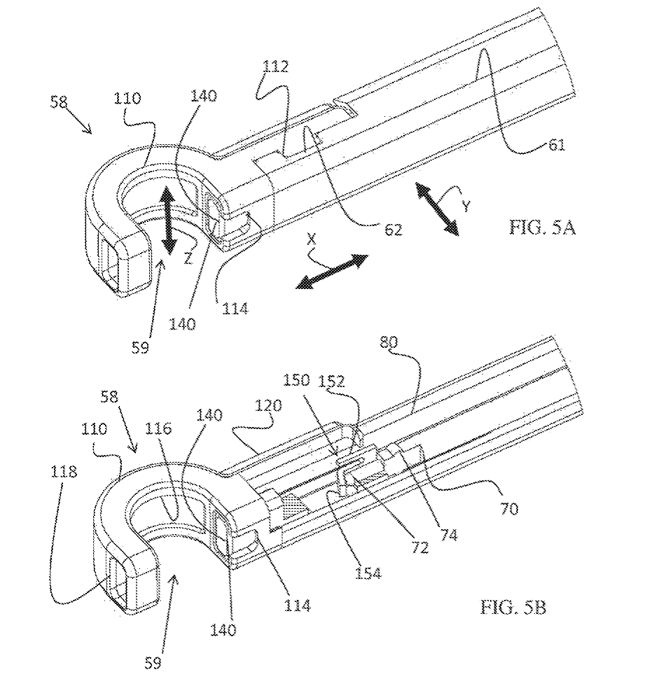

In some embodiments, device 20 may be structured and/or include structures and depicted schematically in FIGS. 4A-6F. FIG. 3A, for example shows a perspective view of device 300 having a blood vessel binding member 50, which is shown in greater detail for example in FIGS. 4B and 5A and 5B. Blood vessel binding member 50 comprises an open-frame shaped member 51 defining a gap 59 for accepting at least one blood vessel, and an adjustable frame-completing member 140 configured to form, together with the open-frame shaped member 51, an essentially closed frame around the at least one blood vessel.

Blood vessel binding member 50, in the shown example, also comprises an adjustable gap filling member 130 positioned to adjust gap 59 within the frame such that a pressure exerted on the at least one artery is changed.

In this example, open-frame shaped member 58 is essentially is curved. This portion comprises a band cover 120 and a band base 110 structured to accept between them at least a portion of an inflatable member 130. Inflatable member 130 comprises or is associated with tubing 1301. When connected together, band cover 120 and a band base 110 form a furrow shaped open frame, providing radial support to an adjustable gap filling member positioned within the frame. The furrow may support inflatable member 130 at least from the side distal from gap 59 and at least partially on the lateral sides, thereby directing the inflation of inflatable member 130 towards filling the gap.

Shaft 60 is shown at some detail in FIG. 4C. As seen, shaft 60 comprises a shaft chassis 61 which may serve to support and/or hold together and/or frame shaft components as shown. In this example, the shaft comprises two elongated rods, namely plunger 70 and support rod 80. One or both of the rods may be used to provide the shaft with some rigidity so as to enable its directing blood vessel binding member 50 through the body. In some embodiments, only a single elongated rod is used, while in others, two or more rods may be incorporated in the shaft. One or more elongated rods may include portion of a lock/release structure, as is exemplified below.

Attention is now drawn to FIGS. 5A and 5B showing a distal portion of a device outlined by dashed line A in FIG. 4A. In FIG. 5A open frame shaped member 58 is shown attached to chassis 61 and the frame shape is open. A connector 62 is schematically shown holding the construct in place and restraining movement of the frame shape and shaft one with respect to the other along the marked X, Y and Z axes. Plunger 70 and support rod 80 are moveable along the X axis. Chassis 60 is structure with an opening along its length, so that it may be may be disconnected from other components of the device (e.g. the implantable portion comprising frame shaped member 58) by being moved along the Y axis and/or using radial rotation. Frame-completing member 140 is essentially bar shaped and retracted into the chassis 61, such that the frame shape is open. The position of frame-completing member 140 is adjustable along the X axis, to allow closing the frame shape, by moving plunger 70.

In a partially exploded view (FIG. 5B) parts of band base 110, frame-completing member 140 and chassis 61 are partially sectioned to enable viewing some components of lock and release mechanisms. Parts of the lock/release mechanisms are also shown in FIG. 5C. Clip 150 comprises a tongue 154 between two arms 152. Clip 150 may be made for example of spring metal sheet (stainless steel or Nitinol), and may be welded to the frame-completing member 140 along it arm 152 (both sides). Plunger 70 comprises a pushing ram 72 and a bayonet protrusion 74.

Before and during implantation, at the frame shape is being pushed to its position in the body, it is desired to maintain it in an open position. Alternatively, the frame shape may be pushed through the body in a closed position and opened (e.g. using plunger 70) at or near the blood vessel. In the instant example, before closing the frame shape, pushing ram 72 is positioned such that tongue 154 is inclined forward towards frame-completing member 140 thereby holding it in an unlocked position. Additionally, bayonet protrusion 74 is positioned next to a matching recess (not shown) thereby locking plunger 70 in its position so that it may not inadvertently detach from the device nor cause frame-completing member 140 to shift its position. To release plunger 70, it must be rotated along its longitudinal axis, to one of two positions: in one position, plunger 70 remains attached to the device and is capable of moving frame-completing member 140 along the X axis and into a locked position.

When frame-completing member 140 has been pushed by pushing ram 72 to the proper closed position, tongue 154 is in position near a matching recess. As plunger 70 is retracted, tongue 154 descends into the recess 114 thereby locking the frame shaped in a closed position. Optionally, removal of the device includes introduction of a shaft 60 having a plunger 70 into the device so as to release tongue 154.

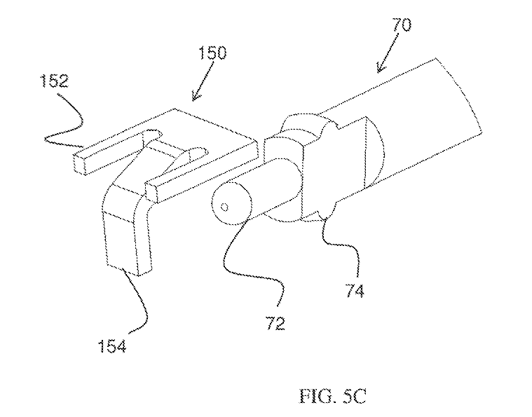

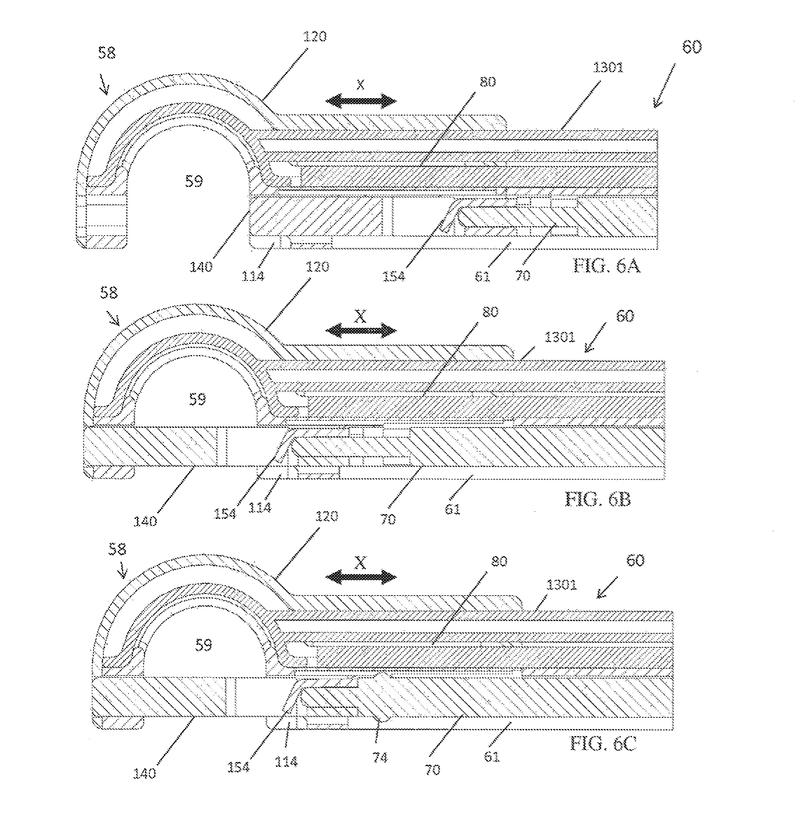

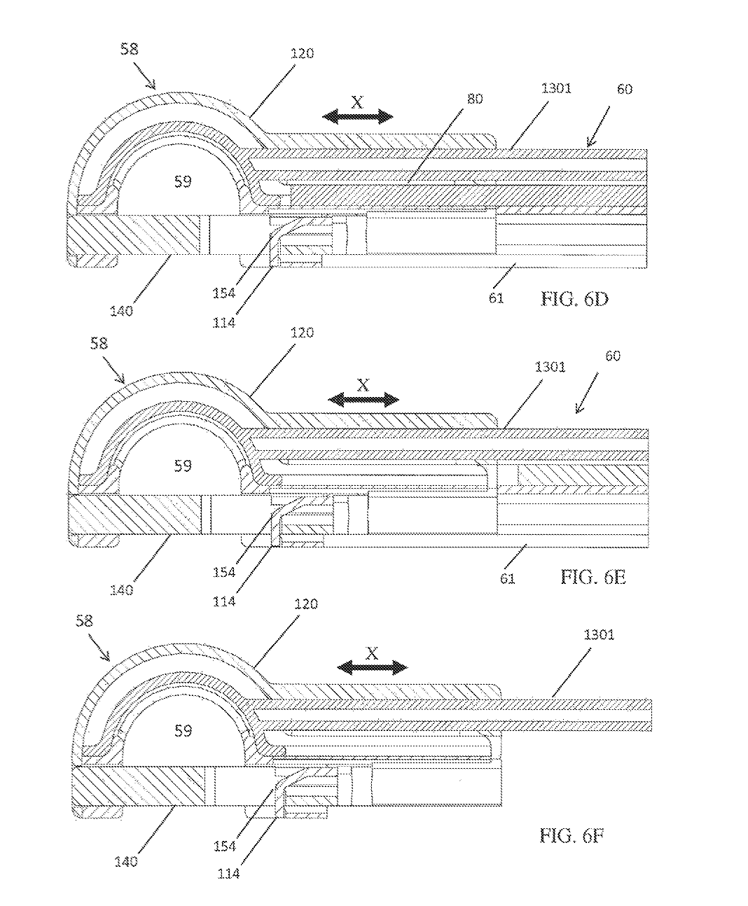

Shaft 60 and/or some components thereof may be removed from the implantable portion of the device, which may comprise for example the implantable portion 400 as shown in FIG. 4B. Shaft 60 and/or some components thereof may optionally be used to remove the implantable portions at a later time. An example for some lock/release structures associated with the attachment and/or removal of the shaft and its components from an implantable portion of the device and/or in the locking in position of a frame-completing member are shown by way of example in FIGS. 6A-6F, depicting a cross sectional through a portion of a device 300 as outlined by dashed line A in FIG. 4A at different stages during implantation and extraction of a device according to some embodiments of the invention.

FIG. 6A shows the device with a shaft 60 attached and the open frame shaped member 58 being open, with frame-completing member 140 retracted, and gap 59 being ready for accepting or removing the at least one blood vessel. This may be for example during the device's shelf life and/or when the device is being moved in and/or out of a patient's body. As seen, tongue 154 is in a lifted position, being slightly raised by plunger 70. Plunger 70 is locked in position by bayonet protrusion 74 and matching structures that prevent its movement along the X axis (not shown). Tubing 1301 and support rod 80 are also seen within chassis 61. When in this configuration, the open-frame shaped region 58 may be positioned in a patient's body such that the at least one blood vessel passes through gap 59.

In FIG. 6B—plunger 70 is pushed forward along the X axis thereby pushing frame-completing member 140 into its closed position and closing frame shaped member 58 around gap 59. In this position, tongue 154 is still slightly raised but is already in position adjacent recess 114. In FIG. 6C—plunger 70 was rotated around the X axis, thereby releasing bayonet protrusion 74 (now visible). This enables extraction of plunger 70 by pulling it along the X axis and away from frame shaped member 58. The steps depicted by FIGS. 6B and 6C may for example be performed during surgery, once the device is positioned such that at least blood vessel passes through gap 59.

FIG. 6D shows the frame shaped member 58 in a closed configuration with plunger 70 already pulled out. Tongue 154 is locked into recess 114 thereby locking frame-completing member 140 in position, to prevent inadvertent opening of the frame and premature disengagement of the at least one blood vessel.

Finally, in FIGS. 6E and 6F other positions of shaft 60 are removed. In a one step (FIG. 6E), support rod 80 is pulled out similarly to plunger 70. As a safety feature, this may be prevented by one or more lock/release structures, which may necessitate for example prior removal of plunger 70 and/or rotational movement of support rod 80. This, in turn, may enable removal of chassis 71 and/or other components of the device that are not to be implanted. FIG. 6F depicts only implantable portions of the device, comprising frame shaped member 58 and tubing 1301 which may be connected to an implanted or external reservoir and/or controller and/or to an port accessible from outside the patient's body.

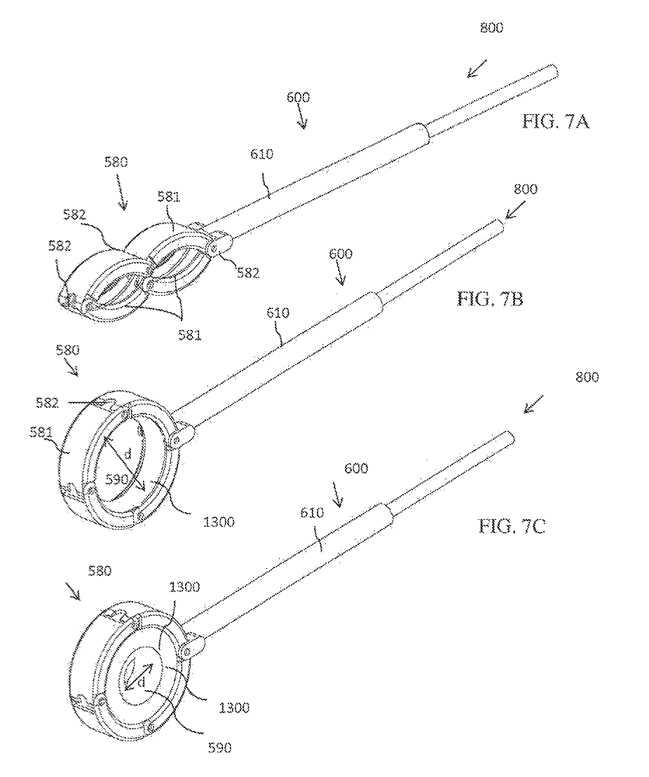

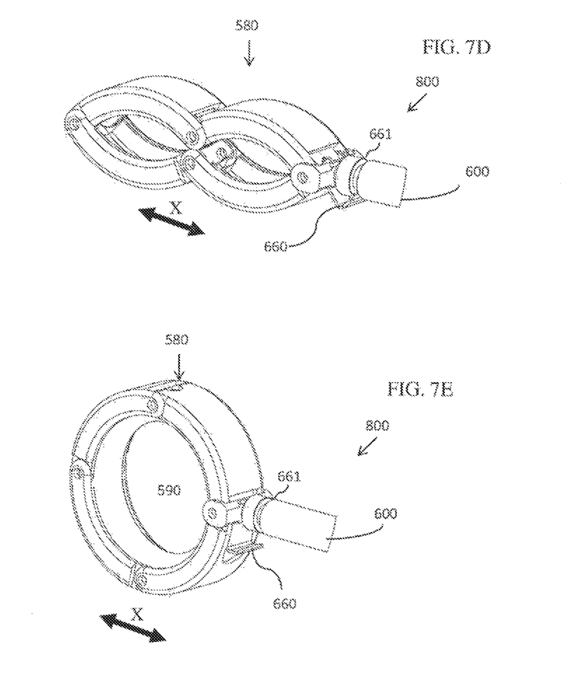

In some embodiments, system 200 may comprise a device 800 as shown schematically in FIGS. 7A-7E. Device 800 comprises a shaft 600 attached to a frame shaped member 580 defining a gap 590 for accepting at least one blood vessel. Frame shaped member 580 may have least an expanded configuration (as shown in FIGS. 7B and 7C) and a collapsed configuration (as shown in FIG. 7A). The device 800 further comprises an adjustable gap filling member, shown here as an inflatable member 1300 associated with the frame shaped member 580 and positioned to adjust gap 590 within such that a pressure exerted on the at least one blood vessel is changed. For example, frame shaped member 580 may have a furrow shape surrounding the circumference of gap 590 and inflatable member 1300 may be positioned around the gap within the furrow.

During implantation and/or extraction of an implantable portion of device 800, frame shaped member 580 may be in a collapsed formation with the inflatable member 1300 deflated or inflated only partially such that frame shaped member 580 is in a collapsed configuration (FIG. 7A). This inflation status of inflatable member 1300 is termed a collapse inflated status. Frame shaped member 580 may comprise a plurality of linked rigid units 581 (in this example four arched shaped units) with interspersed separators 582. The separators 582 may include one or more of hinge structures and/or flexible units that would allow frame shaped member 580 at least to change from a collapsed configuration to an expanded one. Optionally, separators 582 also allow frame shaped member 580 to collapse for removal.

Once frame shaped member 580 is at a position near the at least one blood vessel, inflatable member 1300 is slightly inflated (e.g. by moving a fluid via a tube associated with shaft 600 into the inflatable member) to assume a partially inflation status. This causes units 581 to adjust such that frame shaped member assumes an expanded configuration.

Optionally, frame shaped member 580 is sized and shaped so that a whole ovary may be passed through gap 590 when the frame shaped member 580 is in an expanded configuration without causing significant damage to the ovary, while the ovary is within the body and connected to the body by at least a portion of a ligament and at least one blood vessel.

Once the frame shaped member 580 is in position with the at least one blood vessel passing through gap 590, inflatable member 1300 may be inflated further to have one or more high inflated statuses having a reduced size d of gap 590. In a high inflated status, some pressure is exerted by inflatable member 1300 on the one or more blood vessels within gap 590 and by controlling the degree of inflation such pressure may be controlled as disclosed herein. A high inflated status may also serve to maintain the blood vessel binding member in location (in addition or instead of additional supporting structures such as stitches, biological adhesives and the like).

The inflation status of inflatable member 1300 may also be used to control a lock/release structure which is used to lock shaft 600 and/or any of its components (e.g. a support rod and chassis 610) to frame shaped member 580. An example for this is depicted in FIGS. 7D and 7E. In FIG. 7D, a latch 660 is seen in association with blood vessel binding member 580, which is shown in a collapsed configuration. In this configuration, latch 660 snaps into groove 661 of shaft 600. This prevents or at least encumbers inadvertent dissociation of the shaft 600 from blood vessel binding member 661 (e.g. by rotating around the X axis). As inflatable member 1300 inflates sufficiently, the blood vessel binding member 580 expands and latch 660 disengages from groove 661, thereby releasing the lock/release structure, and enabling the detachment of shaft 600 and/or any of its components. As a safety measure, the degree of inflation that is required for removal of shaft 600 may optionally be set to be at least a minimal pressure that is needed to at least partially occlude the at least one blood vessel. In operation, inflatable member 1300 may be inflated and/or deflated as necessary so as to control the size d of gap 590 and/or to regulate the pressure exerted on the at least one blood vessel within the gap.

In the description and claims of the present application, each of the verbs, “comprise”, “include” and “have”, and conjugates thereof, are used to indicate that the object or objects of the verb are not necessarily a complete listing of components, elements, or parts of the subject or subjects of the verb.

Descriptions of embodiments of the invention in the present application are provided by way of example and are not intended to limit the scope of the invention. The described embodiments comprise diff rent features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A method for protecting at least one gonad from a blood borne cytotoxic drug, the method comprising:
reducing blood flow to at least one gonad of a patient undergoing cytotoxic treatment for an occlusion time interval; and
allowing blood flow to the at least one gonad to resume after the occlusion time interval.

2. The method of claim 1, wherein the occlusion time interval at least partially overlaps with a time when the blood borne cytotoxic drug is present in the blood system of the patient.

3. (canceled)

4. The method of claim 1, including:
administering the blood borne cytotoxic drug to the patient for drug administration time interval.

5. The method of claim 4, wherein the occlusion time interval covers a period of time after the end of the drug administration time interval.

6.-8. (canceled)

9. The method of claim 1, wherein the occlusion time interval is 24 hours or less.

10. (canceled)

11. The method of claim 1, comprising:
stopping blood flow to the at least one gonad during at least a portion of the occlusion time interval.

12. The method of claim 1, comprising applying an ischemia reducing treatment to the at least one gonad.

13.-18. (canceled)

19. The method of claim 1, comprising:
allowing partial blood flow to the at least one gonad during at least a portion of the occlusion time interval.

20. The method of claim 1, wherein reducing blood flow includes intermittent stopping of blood flow to the at least one gonad.

21. The method of claim 1, wherein reducing blood flow includes reducing rate of blood flow.

22. The method of claim 1, comprising:
implanting a device in association with a blood vessel supplying blood to and/or taking blood from the at least one gonad;
wherein reducing blood flow and allowing blood flow to resume are performed by the device.

23.-27. (canceled)

28. A system for protecting at least one gonad from a blood borne cytotoxic drug, the system comprising:
an implantable device comprising a blood vessel binding member comprising a gap having an adjustable width and configured for accepting through it at least one blood vessel supplying blood to and/or taking blood from the at least one gonad; and
a control interface for adjusting the gap such that a pressure exerted on the at least one blood vessel is changed.

29. The system of claim 28, wherein the gap may be reduced to a degree that would stop blood flow to the at least one gonad.

30.-32. (canceled)

33. The system of claim 28, wherein the blood vessel biding member comprises an inflatable portion.

34.-36. (canceled)

37. The system of claim 28, comprising a controller for controlling the adjusting.

38.-39. (canceled)

40. The system of claim 37, wherein the controller is configured to reduce the gap for an occlusion time interval and increase the gap at the end of the occlusion time interval.

41.-45. (canceled)

45. The system of claim 37, comprising:
a drug administration sensor configured for being associated with a drug administering device and for providing data relating to the administration of a cytotoxic drug;
wherein the controller is configured to receive said data from the drug administration sensor, and to control the adjustment based on the data.

46. The system of claim 37, comprising:
an ischemia sensor for sensing an ischemia related parameter at the at least one gonad;
wherein the controller is configured to receive said parameter from the ischemia sensor, and to control the adjustment based on the parameter.

47. The system of claim 37, comprising:
a blood flow related sensor for sensing a blood flow related measure relating to the at least one gonad;
wherein the controller is configured to receive said measure from the blood flow related sensor, and to control the adjustment based on the measure.

48. (canceled)

49. The system of claim 47, wherein the controller is associated with an interface for receiving at least one treatment specific parameter and the controller is configured to select an operation protocol according to the at least one treatment specific parameter.

50.-86. (canceled)
